(19) United States
(12) Patent Application Publication
(10) Pub. No.: US 2022/0401269 A1
(43) Pub. Date: Dec. 22, 2022

(54) MOISTURE MONITORING SYSTEM FOR ABSORBENT ARTICLES

(71) Applicant: P2SOLUTIONS, Aalst (BE)

(72) Inventor: Bjorn VAN KEYMEULEN, Aalst (BE)

(73) Assignee: P2SOLUTIONS, Aalst (BE)

(21) Appl. No.: 17/765,575

(22) PCT Filed: Oct. 1, 2020
(86) PCT No.: PCT/EP2020/077562

§ 371 Date: Mar. 31, 2022

(30) Foreign Application Priority Data
Oct. 1, 2019 (BE) BE2019/5645

(51) Int. Cl. A61F 13/42; A61L 15/58

(52) U.S. Cl. CPC A61F 13/42; A61L 15/58; A61F 2013/424

(57) ABSTRACT

The invention relates to an improved moisture monitoring system for absorbent articles, characterized by its simple design, application and use, and easy electrical interconnection. The moisture monitoring system appears as a strip being fixed onto the outer surface of an absorbent article. The invention also relates to absorbent articles comprising such moisture monitoring system, and to a method for applying such moisture monitoring system onto an absorbent article.

### TECHNICAL FIELD

The invention relates to an improved moisture monitoring system for absorbent articles. Such moisture monitoring system while avoiding leakages is characterized by its simple design, application and use, and easy electrical interconnection. The moisture monitoring system is particularly provided onto the outer (exterior) surface of an absorbent article, i.e. away from a person wearing such an absorbent article. Moreover, the moisture monitoring system appears as a strip being fixed or attached onto the absorbent article. The invention also relates to absorbent articles comprising such moisture monitoring system, and to a method for applying such moisture monitoring system onto an absorbent article.

### BACKGROUND OF THE INVENTION

Absorbent articles such as diapers or sanitary pads for instance, are yet available having a sensor system or a kind of intelligence for monitoring contamination and/or detecting leakages therein, hence so-called smart diapers. Moisture sensors are typically used for indicating a degree of contamination, and possibly also measuring or determining the amount of contamination or more specifically the volume of urine that is absorbed. Volume measurement systems and methods are disclosed in the art using conductive sensors and electrode systems.

Volume monitoring systems comprising a sensor system may comprise a conductive circuit, i.e.

material with electrical properties, more in particular the conductive circuit may comprise electrodes, e.g. moisture (volume) sensing electrodes. The conductive circuit is characterized by resistance values or -more generically speaking -impedance values representing respectively an adjusted resistance or impedance design, as defined by a (mathematical) distribution in the variation of respective resistance or impedance values over the area of the absorbent article.

Current diaper moisture sensors are generally based on direct contact between urine, being absorbed by a mixture of fluff and superabsorbent polymer (SAP), and the (e.g. printed) conductive sensors. While referring to a resistance measurement system, urine on the sensor leads to a lower resistance being measured. In other words, the resistance value captured by the sensor, is in relation to a certain extent to the liquid (or urine) intake of the diaper. Resistance decreases where the absorbent article becomes wet, i.e. where the liquid (or urine) is absorbed within the diaper. Or else, the increase in liquid (or urine) causes the electrical resistance between the sensor electrodes to decrease.

Most of the diapers having moisture sensors, have their sensors provided at or close to the inner surface of the diaper, i.e. towards a person wearing such a diaper. The sensors can also be embedded for instance, just below the inner surface. Measurements are hence supposed to be more precise and more accurate whereas the distance to the person wearing the diaper, and thus to where the liquid (or urine) will flow, is the closest possible. Moreover, except maybe for the inner surface layer of the diaper, there are no physical barriers between sensing system and liquid flow, hence not disturbing the measurement either. Such interior measurement systems, as can be further referred to because of measuring at the interior area of the diaper, more particularly the sensors being used therefore, are however difficult to design and to apply within the diaper. Moreover, the electrical interconnection between the sensors on one hand, and the measurement and transmission unit on the other hand can be fairly complex or poor in connecting quality. In other words, interior systems do have their drawbacks. An exterior monitoring system, meaning having the sensors provided at the outer surface of the diaper, can be thought of for eliminating the disadvantages of the interior application.

Examples do exist in the art wherein a moisture, vapour or humidity sensor is arranged on an outer layer of the diaper. In most of these cases the sensor is applied either at the front just below abdomen level or else being very locally provided, and hence not covering the entire wetting surface or range of the diaper. For instance, US patent application U52018085262 A discloses a system for remotely monitoring the status of wetness of a diaper worn by a patient using a reusable electronic vapor sensor, wherein the system may comprise a pouch portion having the sensor included, being located on the outer surface of the diaper. Such pouch portion however, is only monitoring locally at a certain place where it is (together with the sensor) installed. The idea of having a bag or pocket provided outside the diaper but attached thereto, and wherein the bag or pocket comprises the moisture sensor for performing the measurements or monitoring leakages, is also described in utility model CN208942589 U, further referring to such bag as a kind of accessory attachment for storage of e.g. detection accessories. In the international patent application WO2012166765 Al a sensor system is described wherein multiple sensors are separable from or removably mounted onto the exterior garment-facing surface of the absorbent article. One or more stimuli are detected with multiple elements at multiple locations. The sensors being provided in different areas, delivers a widespread though rather unpractical and cumbersome solution. Moreover, only local spots of sensors being used here, instead of extended or elongated strips. References can be mentioned illustrating longitudinally extending conductive zones or elongated sensors mounted to the outer cover of the article, such as described in US2010164733 A or US2008058740 A, or as in JP2017189348 A applied onto the exterior surface of the backsheet. In any of these, the conductive traces or sensors are directly formed on the outer surface of the absorbent article, implying for example difficulty in production e.g. fast and simple attachment, connecting issues and difficulty in (accuracy of) measurement, and likely discomfort for the person wearing the absorbent article. A substrate or additional layers with the conductive circuit or electrodes printed thereon, can be thought of for an improved manufacturing, for which can be referred to for example to WO2018229017 Al or WO2015003712 Al. However, in WO2018229017 Al the substrate is to be incorporated within the absorbent article, and not be mounted thereon outside, leading to different features and functionalities than required for exterior use. The stacking of layers wherein electrodes and dielectrics are sandwiched as in WO2015003712 Al, discloses a retrofit solution to be placed around the absorbing material in an absorbent article covering part of the interior and exterior surface, again resulting in different specifications than when intended for exterior use only. The process of manufacturing sensing elements by spraying conductive inks on moving sheet of layer is for example described in US2019240080 A, here particularly to be applied on the interior side of the absorbent article's bottom impermeable layer or any surface of its top permeable layer.

Reference can also be made to smart diapers with alternative measurement techniques, i.e. not using a moisture sensor but a different type of sensing or detection for leakages, wherein the sensor system is provided on the outer surface or fixed to the outside layer of the diaper. Amongst those, some suggest the use of a rather large or extended sensing area, although, because of the different technique in detecting liquid, the principles here cannot simply be adopted to the moisture monitoring system with moisture sensor.

There is a need for a moisture monitoring system for absorbent articles with simple solution to apply a sensor system comprising a large and/or extended surface detection range, and obtaining good and reliable electrical interconnections, as well as a stable mechanical design.

Aim of the invention

The aim of the invention is to provide a moisture monitoring system for absorbent articles with easily applicable sensor system, preferably mounted onto its exterior or outer surface, comprising a large and/or extended surface detection range. The aim is also to obtain good and reliable electrical interconnections between the sensor system, and measurement and transmission unit, and a stable mechanical construction of the moisture monitoring system.

### SUMMARY OF THE INVENTION

In a first aspect of the invention a moisture monitoring system is provided, for use in connection with an absorbent article, wherein the moisture monitoring system comprises a sheet having a first and a second surface. Such sheet comprises for example of PE, PP or PET. According to an embodiment the position of the sheet is for instance such that the first surface is towards the absorbent article to be assembled with, whereas the second surface is heading the outer environment, i.e. one could also refer to as garment-facing surface. The moisture monitoring system also comprises a sensor system being provided onto the first surface (e.g. heading the absorbent article or person wearing it) of the sheet, more in particular onto a part thereof being for example the central area of the first surface, and such part also referred to as first part of the sheet's first surface. The sensor system comprises of printed sensors (or electrodes) made of conductive (ink) traces, and may comprise of sensors such as moisture sensors. The moisture monitoring system further comprises a measurement unit being provided with electrical interconnections, wherein this measurement unit is to be mounted onto and connected with the sensor system provided onto the sheet, by means of the electrical interconnections. Via the electrical connections provided thereon, the measurement unit being at least in part physically connected with the sensor system. According to an embodiment, the measurement unit is designed with dimensions for being fit within the borders of what is defined as the sheet's first surface, preferably within the central area thereof, and covering the sensor system with which it is being connected by means of the electrical interconnections. The moisture monitoring system is for example to be provided along the length of the absorbent article, and further referred to as longitudinally oriented, e.g. in the appearance of an elongated printed sensor strip, and to be permanently fixed onto the absorbent article, more in particular onto its outer surface, by means of an adhesive such as hot melt glue. According to an embodiment, the moisture monitoring system resembles as a longitudinally oriented and elongated printed sensor strip comprising of an elongated sheet onto which conductive traces (of the sensor), e.g. longitudinally oriented and elongated electrodes made of conductive ink, are applied, to be electrically connected with a measurement unit, located between the sensor traces and the absorbent articles outer surface. By means of the electrical connections provided onto the measurement unit, the measurement unit is at least in part physically connected with the conductive traces (of the sensor), meaning that the electrical connections are for example in physical contact with part of the elongated electrodes or conductive traces (such that the electrical connection as needed is made) but not necessarily being in physical contact with the entire length of the elongated sensor traces. The printed sensor strip may herewith represent an elongated (circuit) pattern within the central area, here elongated central area, of the sheet's first surface. The moisture monitoring system may be provided along the absorbent article's entire length.

Whereas the sensor system of the moisture monitoring system is to be provided on a first part of the sheet's first surface, the adhesive can be provided onto a second part of the sheet's first surface. Such second part is for instance at the outer edge of the first surface. Providing the adhesive onto this second part can be done for example by directly applying the adhesive thereon. However, according to an embodiment, this can also be done in an indirect manner, e.g. by indirectly bringing the adhesive onto this second part, when this adhesive is for instance originally applied onto the outer surface, also called backsheet, of the absorbent article. By bringing moisture monitoring system (or, for example, sensor strip) and absorbent article (or, for example, diaper) together, the adhesive (or, for example, the glue) gets from one to the other and both can be attached to each other or fixed permanently.

In a second aspect of the invention an absorbent article is provided, comprising an outer surface, and attached thereto a moisture monitoring system in accordance with first aspect.

In a third aspect of the invention a method is provided for applying the moisture monitoring system in accordance with first aspect onto an absorbent article's outer surface, and along its length, comprising the steps of (i) providing a sheet comprising a first and a second surface; (ii) providing a sensor system comprising of printed conductive traces onto a first part of the first surface of the sheet; (iii) applying an adhesive onto a second part of the first surface of the sheet; (iv) connecting a measurement unit comprising electrical interconnections with the sensor system by means of the electrical interconnections; (v) connecting the adhesive with the outer surface of the absorbent article. According to an embodiment, the method comprises a further step (vi) of covering the moisture monitoring system and its periphery of or onto the absorbent article with a cover layer, meaning that the entire moisture monitoring system including its surrounding surface area being part of the absorbent article is covered with an additional layer.

According to an embodiment, an alternative method is provided for applying the moisture monitoring system in accordance with first aspect to an absorbent article comprising an outer surface, having in step (iii) an adhesive applied onto outer surface of the absorbent article, to be connected in step (v) with a second part of the first surface of the sheet such that strip or more in particular its sheet is fixed with the absorbent article, more in particular with its outer surface or so-called backsheet.

### BRIEF DESCRIPTION OF THE DRAWINGS

FIG. 1 illustrates schematically cross sectional view of an embodiment of the moisture monitoring system attached to an absorbent article worn by a person, in accordance with the invention.

FIG. 2 illustrates schematically further detailed embodiment in cross sectional view of the moisture monitoring system attached to an absorbent article worn by a person, in accordance with the invention.

FIG. 3 illustrates schematically top view of an embodiment of an absorbent article with moisture monitoring system, in accordance with the invention.

FIG. 4 shows picture image of sensor examples for the moisture monitoring system, in accordance with the invention.

FIG. 5 represents a flow-chart embodiment for the method for applying a moisture monitoring system onto an absorbent article, in accordance with the invention.

FIG. 6 illustrates schematically another embodiment in cross sectional view of the moisture monitoring system attached to an absorbent article worn by a person, in accordance with the invention.

FIG. 7 illustrates measurement circuit for moisture volume measurement, also called differential volume measurement, as known in the art, to be applied with the moisture monitoring system in accordance with the invention.

FIG. 8 illustrates schematically how a smart incontinence product can be used in real life in a nursing facility, in accordance with the invention.

FIG. 9 illustrates schematically a variant of the embodiment of FIG. 2 wherein the moisture monitoring system being closely attached to the absorbent article worn by a person, the absorbent article herewith comprising a pocket encapsulating the measurement unit, in accordance with the invention.

### DETAILED DESCRIPTION OF THE INVENTION

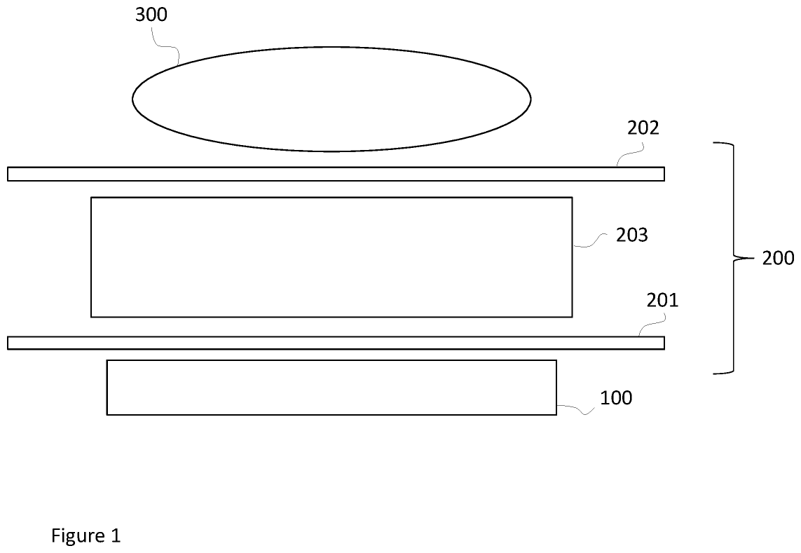

The invention relates to a moisture monitoring system for absorbent articles, amongst which for example diapers, incontinence pads, sanitary napkins or towels and/or other hygienic cloths or similar hygiene products. The moisture monitoring system is designed for avoiding leakages in the absorbent article and is particularly provided onto the outer surface of the absorbent article, meaning away from a person wearing the absorbent article. According to an embodiment, the moisture monitoring system 100 for use in connection with an absorbent article 200 to be worn by a person 300, is schematically depicted in cross sectional view in FIG. 1. The absorbent article 200 comprises of a so-called inner or interior surface layer 202 to be brought in direct contact with the person 300 wearing the absorbent article 200. The inner or interior surface layer 202 is also referred to as top sheet of the absorbent article 200. The absorbent article 200 also comprises of a so-called outer or exterior surface layer 201 further away from the person 300 wearing the absorbent article 200. The outer or exterior surface layer 201 is also referred to as backsheet of the absorbent article 200. In between both surface layers 201, 202, or in other words in between top sheet and backsheet, a filling material 203 is provided, comprising for example a mixture of fluff and superabsorbent polymer (SAP). The moisture monitoring system 100 is provided onto the absorbent article 200, more in particular onto its outer or exterior surface layer 201, or else onto the backsheet of the absorbent article 200. According to an embodiment, the moisture monitoring system 100 is permanently fixed or attached onto the absorbent article 200, e.g. by means of an adhesive such as glue, although a removable version using for instance a pressure sensitive adhesive as is used for release liner products such as e.g. sanitary pads, is not excluded from this invention.

According to an embodiment, a retrofit solution for current existing absorbent articles is also proposed with the present invention. The moisture monitoring system in accordance with the invention, is for example made standalone, being provided with an adhesive layer protected for or before use with a cover layer. Such adhesive layer can be either for permanent fixation, or else for removable attachment. The moisture monitoring system is for instance made and supplied as a strip pre-cut in suitable shape to be directly applied onto a diaper or other type of absorbent article, by the person wearing the diaper or a nursing assistant. Hence such strip attachment is to apply yourself. Instead of a pre-cut strip, one could also think of a strip roll, of which the desired length and/or shape strip is cut depending on the absorbent article onto which the moisture monitoring system is to be applied.

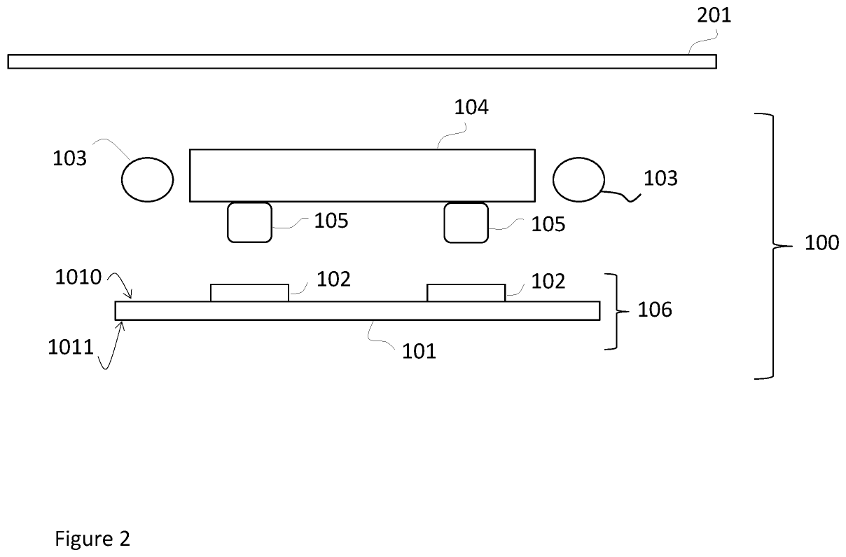

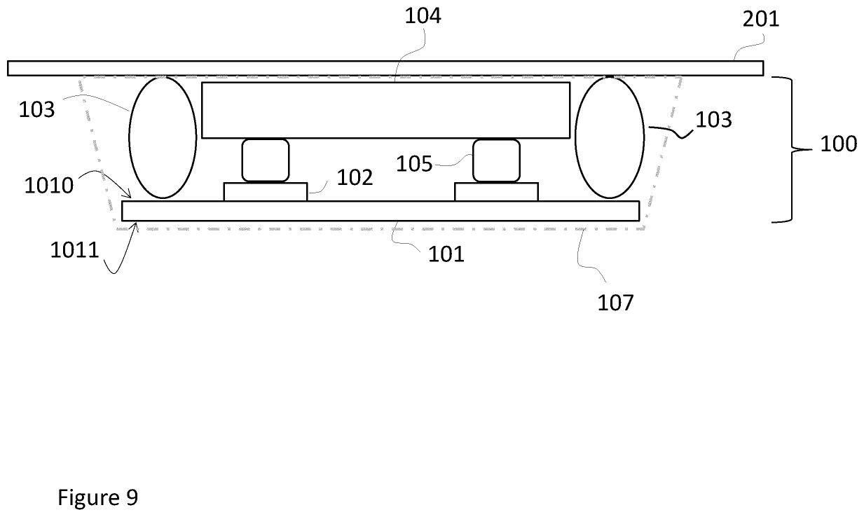

With the embodiment of FIG. 2, more detail of the moisture monitoring system 100 is presented, and herewith the working principle is described. The moisture monitoring system 100 is again provided onto the outer layer or backsheet 201 of the absorbent article 200, and the different components of the moisture monitoring system 200 are now also shown. According to the embodiment, the moisture monitoring system 100 comprises of a sheet 101 onto which a sensor system 102 is provided. With the sheet 101 of the moisture monitoring system 100, two sides or surfaces 1010, 1011 are indicated. One could recognize an inner side or surface 1010 and an outer side or surface 1011. The sensor system 102 is provided onto one side or surface, here the inner side or surface 1010 of the two sides or surfaces of the sheet 101. The side or surface 1010 of the sheet 101 onto which the sensor system 102 is applied, being the side or surface towards the backsheet 201 of the absorbent article 200. Together, this sheet 101 and sensor system 102, can also be referred to as sensor sheet 106. The sensor system 102 is for example determined by printed sensors or more in particular, a printed sensing pattern made of conductive ink. As depicted in FIG. 2, the print or ink of the sensor sheet 106 here being applied onto the side or surface 1010 of the sheet 101 towards the absorbent article 200, or towards the person 300 wearing the absorbent article 200. The type of sensors used are moisture sensors in accordance with the embodiment, although in theory, other types of sensors could also be applicable. The moisture monitoring system 100 further comprises of a measurement unit 104 being provided with electrical interconnections 105 for connecting this measurement unit 104 with the sensor system 102 or e.g. printed sensors of the sensor sheet 106, such that ‘what is sensed’ or the sensor data can be further interpreted by the measurement unit 104. The measurement unit 104 together with its electrical interconnections 105 has dimensions that fit within the gap or space created by the sensor sheet 106, an adhesive 103 applied thereon, and the backsheet 201 of the absorbent article 200 onto which the moisture monitoring system 100 is to be fixed by means of the adhesive 103. The adhesive 103, for example hot melt glue, being particularly applied on (part of) the edge of surface 1010 surrounding the sensor system 102, causing this gap or space and herewith generating a kind of pouch or pocket 107 for the measurement unit 104 to be mounted in, as illustrated in FIG. 9. In other words, the outer dimensions of the measurement unit 104 are designed to fit within the surface area of surface 1010 of the sheet 101. The measurement unit 104 when mounted onto the sensor sheet 106 for being connected therewith by means of the electrical interconnections 105, is covering substantial part of the surface area of surface 1010 of the sheet 101. Moreover, the outer edge area of the surface area of surface 1010 remains uncovered. According to an embodiment, this outer edge area, or part thereof, is provided with an adhesive 103 for attaching or fixing the moisture monitoring system 100 onto the backsheet 201 of the absorbent article 200. For permanent fixing of the moisture monitoring system 100, glue or even hot melt glue can be used as adhesive.

The sheet 101 comprises for example of PE, PP or PET being part of the moisture monitoring system 100 and added onto the standard existing absorbent article structure or design comprising basically top sheet 202 and backsheet 201, and further absorbing substance there in between. Moreover, the sheet 101 is particularly configurated for exterior use, i.e. to be applied onto the exterior or outer surface of the absorbent article 200. As opposed to sheets or substrates provided with sensors or conductive electrodes used in the art, e.g. incorporated within the absorbent article, the sheet 101 may become wet, whereas in case of interior use sheets or substrates have to remain dry and are protected from moisture in order to achieve correct and accurate measurements. In other words, features and functionalities of sheet 101 for exterior use being entirely different from the art's known interior use examples.

The moisture monitoring system 100 introduces indirect moisture measurement by having the moisture sensor or the sensor system 102 provided at the outer surface side 201 or backsheet of the absorbent article 200, and thus not at or close to the inner layer 202 or top sheet of the absorbent article 200 being closest to the person wearing the absorbent article 200 and hence logically resulting in most accurate measurements. The principle or motivation for this indirect measurement -as opposed to direct, (almost) in contact with the person's human body -is based on the fact that backsheet material is breathable nowadays, due to which moisture accumulated within the absorbent article can penetrate towards the outer environment.

A variant of the embodiment of FIG. 2 is depicted in FIG. 9, wherein the moisture monitoring system 100 is illustrated more closely attached to the absorbent article worn by an person. The adhesive 103 provided at least in part between, on the one hand the outer edge of the surface area of surface 1010 of the sheet 101, and on the other hand the absorbent article's outer surface 201, is now encapsulating the measurement unit 104, being mounted between the sheet 101 and the absorbent article 200. As a result, it appears the absorbent article 200 comprises a pouch or pocket 107.

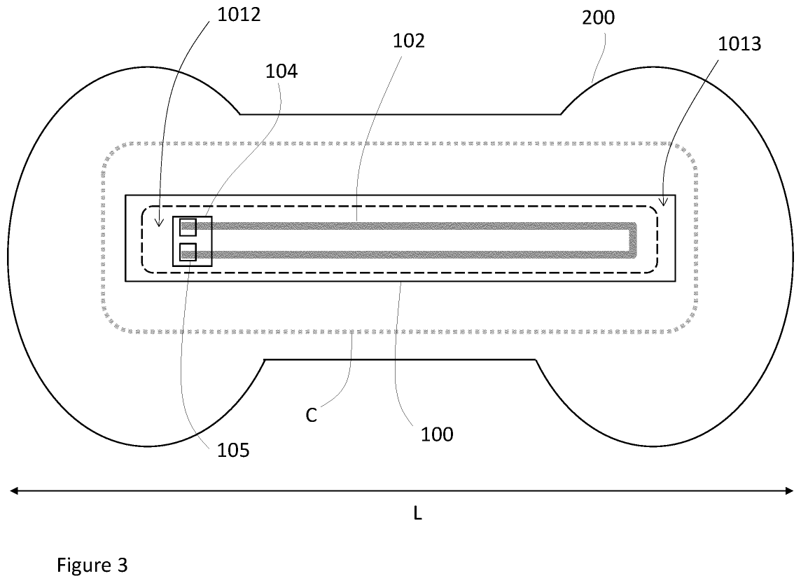

The invention also relates to absorbent articles comprising the moisture monitoring system as described above. FIG. 3 illustrates top view of an absorbent article 200 with moisture monitoring system 100 in accordance with the invention. The moisture monitoring system 100 is herewith represented as a strip in the centre space C and along the length L of the absorbent article 200. The moisture monitoring system 100 itself is further shown with its central area 1012 (within the dashed line), comprising the sensor system 102 or conductive electrodes, and its outer edge 1013 indicated thereon. The electrical connection of the sensor system 102 with the measurement unit 104 is enabled by means of the electrical connections 105. The sensor system 102 or conductive electrodes, are here for some part physically connected with the measurement unit 104, i.e. the outer ends of the conductive electrodes are brought in physical contact with the electrical connections 105 in order to achieve electrical connection with the measurement unit 104. The moisture monitoring system 100 displayed as elongated printed sensor strip with elongated pattern defined by its conductive sensor electrodes measures the moisture penetration effect of the absorbent article 200. By choosing a low conductive printed sensor for the sensor system 102, the evaporated liquid (or urine) from the absorbent article 200 can be measured effectively. According to an embodiment, the basic sheet 101 or substrate of the printed sensor strip is made of material with good adhesion properties particularly for use with hot melt glue as adhesive.

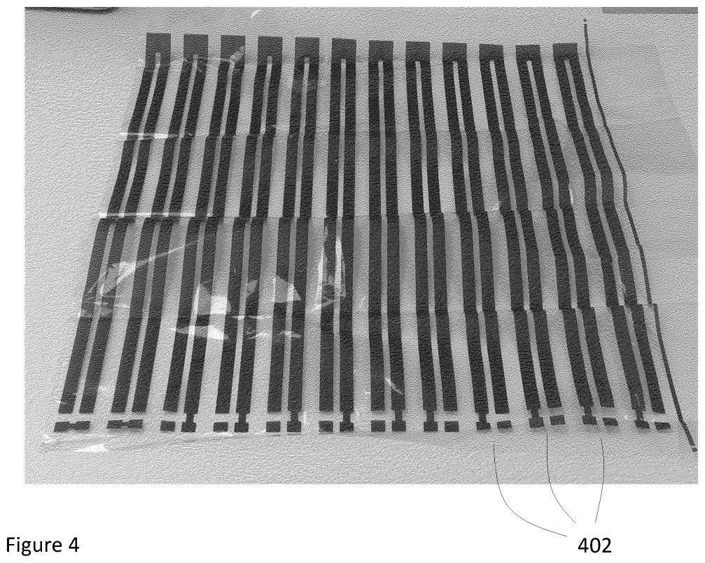

In FIG. 4 a picture image is given of examples of sensors or sensor patterns 402 to be provided within the moisture monitoring system as described above, enabling indirect moisture measurement from the outside layer of the absorbent article. According to an embodiment the sensors or sensor patterns 402 can be printed as a registered or as a non-registered design onto a foil or sheet. Furthermore, next to the sensing part e.g. defined by sensors or sensor patterns 402, an identification part can also be printed on the same sheet. The identification part can be used in order to identify the kind of product (e.g. sensing strip for a diaper), the production batch, etc. According to an embodiment the sensors or sensor patterns and/or the identification part exist of printed conductive ink. According to a further embodiment this ink is applied by screen, gravure or flexo printing. According to an embodiment the resulting sheet resistance after application and curing of the conductive material is between 10 and 100 Ωs/square, between 99 and 1000 Ωs/square, or between 999 and 10000 Ωs/square.

According to an embodiment also non-conductive ink can be applied on the sensor sheet, although not intended then for printing sensing electrodes or other electronic circuitry but rather printing of text, logo's or icons. The usefulness of this is to avoid a print with e.g. branding on the backsheet material. In this way, sensors and commercial prints can be combined in one material layer.

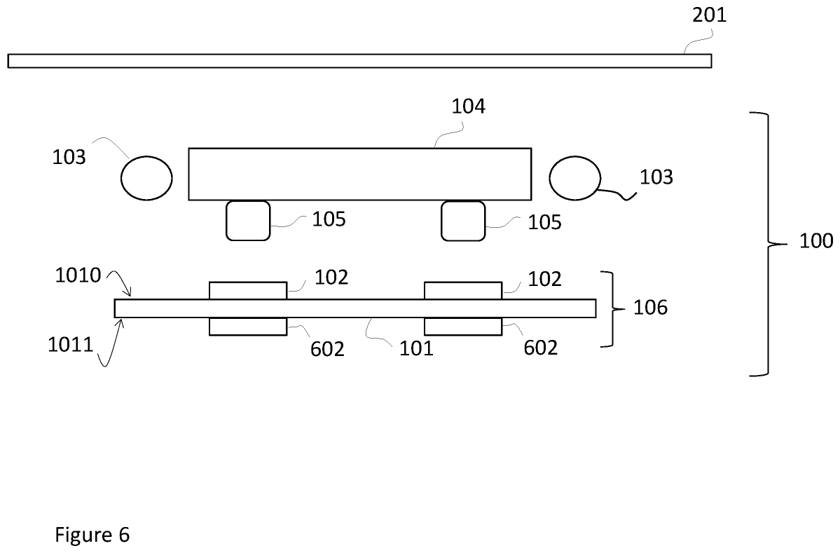

As illustrated in FIG. 6, according to an embodiment additional sensors or an additional sensor pattern 602 can be added to the outer surface or side 1011 of the sheet 101. These additional sensors can be used for example for leakage detection when the pad or diaper starts leaking. When leakage occurs, the surrounding environment and therewith the outer surface part 1011 of the sheet 101, will become wet causing a significant change in measured value of the sensors 602. According to further embodiment the identification part, as mentioned above with the description of FIG. 4, can also be added at the outer side 1011 of the sheet 101 instead of the inner side 1010.

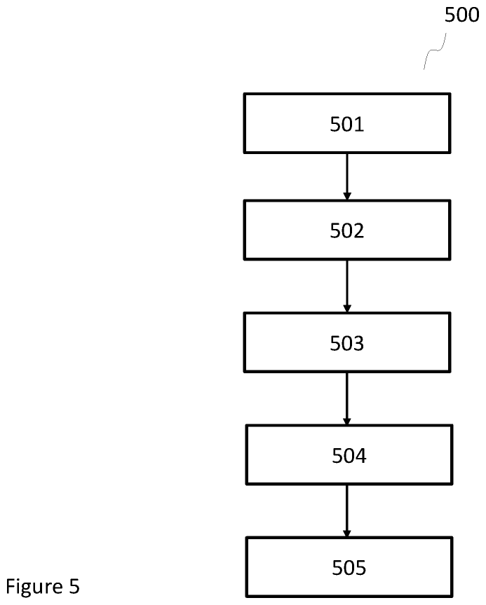

The invention further relates to a method for applying the moisture monitoring system onto an outer surface of an absorbent article. While referring to FIG. 5, representing a flow-chart for the method 500, and to nomenclature of moisture monitoring system 100 and absorbent article 200 as in FIG. 2 and FIG. 3, possible different steps of the method are herewith described. As a first step 501, a sheet 100 is provided, comprising a first 1010 and a second surface 1011. In a second step 502, a sensor system 102 is provided, e.g. printed, onto a first part, for example the central area 1012, of the first surface 1010 of the sheet 101. During a third step 503, an adhesive 103 is applied onto a second part, for example the outer edge 1013, of the first surface 1010 of the sheet 101. According to an embodiment, the adhesive 103 can be applied on sheet 101 for later on fixing or attaching to the backsheet 201 of the absorbent article 200, or else, according to another embodiment, the adhesive 103 can be applied onto the backsheet 201 for later on fixing to or connecting with the strip sheet. In a fourth step 504, a measurement unit 104 comprising electrical interconnections 105 is mounted onto and connected with the sensor system 102 by means of the electrical interconnections 105. As a fifth step 505, the adhesive 103 as applied onto second part of the sheet 101 is connected with the outer surface 201 of the absorbent article 200. According to an embodiment, after the fifth step 505 a further step is also provided during which the moisture monitoring system 100 and its periphery of or onto the absorbent article 200 are covered with a cover layer.

According to an embodiment, the adhesive is a hotmelt material applied with a glue head on a flexible material of the strip sheet 101 or onto the backsheet 201 of the absorbent article 200. According to a further embodiment the glue lines of the adhesive 103 have a width of 3 to 5 mm, 4 to 7 mm or 6 to 10 mm. According to a further embodiment a margin of 0.5 to 2 mm, or of 1 to 5 mm, is taken from the sides of strip sheet 101 to avoid glue artefacts due to misalignment between backsheet layer 201 and strip sheet 101. While referring to FIG. 3, a margin of for example 2 mm is taken from the border of the outer edge 1013, onto which no adhesive is applied. Further away from the border (than this 2 mm) adhesive is then applied, still remaining in outer edge area 1013.

According to an embodiment the sensor sheet 106 has a typical width of maximum 70 mm, typically 60 mm and minimum 10 mm.

The method for applying the moisture monitoring system with sensor system, more in particular the step of connecting a measurement unit comprising electrical interconnections with the sensor system by means of the electrical interconnections, not only results in an electrical connection but moreover leads to a mechanical stable connection of the individual parts. This inherently resulting from the design of the moisture monitoring system, wherein the space that is filled with the measurement unit and nicely closed at the edges with the adhesive, appears as a kind of pouch or pocket.

The moisture monitoring system in accordance with the invention, as described, can be noticed for its simple design and easy way of application or use in connection with an absorbent article. Further, with this moisture monitoring system, recycling afterwards thereof, can be performed easily since all electronics and smart parts of the system are packaged together, and easy to disassemble.

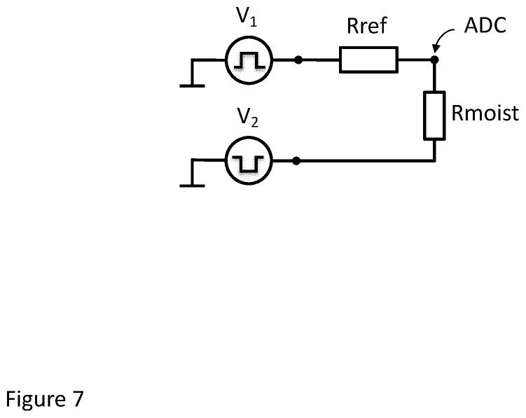

The moisture volume measurement method is now further described with FIG. 7. It shows how one can achieve an inversely applied voltage, also called differential, over the resistive moisture sensor by only applying positive square waves towards the sensor set-up. In other words, the moisture volume measurement is performed by means of a so-called differential measurement approach in order to overcome electrical polarisation of the contamination or more in particular of the contaminating substance e.g. urine, within (or even getting outside of) the absorbent article.

The differential measurement approach is based on the application of a differential or complementary voltage over two separate points in an electrical circuit as known in the art. The measurement circuit of FIG. 7 represents a resistive moisture sensor with resistance Rmoist and a reference resistor with resistance Rref. The resistance of moisture Rmoist is measured by an analog-to-digital convertor (ADC) by using a conventional voltage divider (as known in the art) wherein the reference resistor with resistance Rref is remained constant. The resistance of the moisture sensor will depend on the moisture volume, or else the contamination indicating a certain degree of saturation within the absorbent article.

During the half of the period voltage V1 (e.g. +5V) is high and voltage V2 is low (e.g. OV) so that the voltage over Rmoist is:

$${V}_{Rmoist} = \frac{Rmoist}{Rmoist + Rref} ( V 1 - V 2 ) = \frac{Rmoist}{Rmoist + Rref} \cdot 5 V$$

During the other half of the period voltage V1 (e.g. OV) is low and voltage V2 is high (e.g. +5V) so that the voltage over Rmoist is:

$${V}_{Rmoist} = \frac{Rmoist}{Rmoist + Rref} ( V 1 - V 2 ) = \frac{Rmoist}{Rmoist + Rref} ( - 5 V )$$

This gives us again a negative voltage value, also referred to as differential signal over the terminals of Rmoist in order to avoid polarisation as is further described in patent application WO2018/229280 A1 from the same applicant.

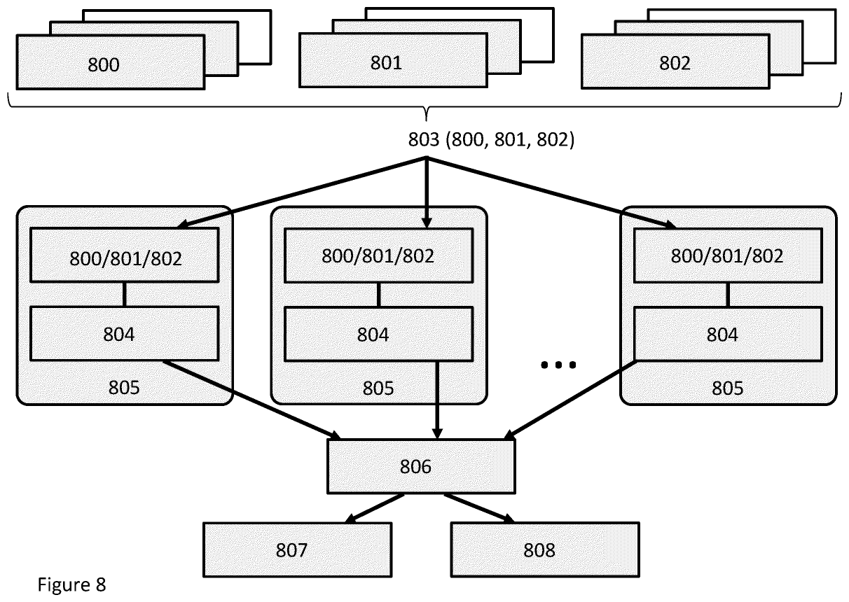

FIG. 8 shows how a smart incontinence product 800, 801, 802 can be used in real life in a nursing facility. In fact three different types of smart incontinence products 800, 801, 802 are shown. The collection 803 is the stock of all smart incontinence products 800, 801, 802. The products 800, 801, 802 can be used by users 805, all respectively wearing a smart incontinence product 800, 801, 802 and respectively having an electronic measurement module 804 attached thereto. This measurement module 804 transmits the measured data wireless to a central processing system 806, which can be the cloud (or remote server), or either can be a local server, or else a combination of a local server and the cloud, etc. The measured data can include:
Product identification: having the product identified and thus knowing which product 800, 801, 802 it is in order to adjust the stock 803 automatically, to make ordering and supplementing of products easier and to overcome manual stock counting. Identification data is originating from the identification part of the strip sheet.
Urine contamination: to identify when and how a product is saturated. By means of this data caregivers can identify the optimal moment to change an incontinence product to overcome leakages. Furthermore, it can be identified if the wearer of the product is using the correct product at the correct moment by reviewing the moment when a product is changed and the remaining capacity of the product. Urine contamination data is originating from the sensors at the inner side of the strip sheet.
Leakage detection: when an incontinence product starts leaking a flag will be set to trigger an immediate action of the caregivers and to overcome skin irritation and other disadvantageous and uncomfortable issues associated with leaking incontinence material. Leakage data is originating from the sensors at the outer side of the strip sheet.

From the central processing system 806, the measured data having been processed is then transmitted to an output system 807, 808 such as for instance a user interface 807 e.g. a tablet or smart phone for a nurse, or a management application 808 for reporting.

1-13. (canceled)

14. A moisture monitoring system for an absorbent article to be applied onto an outer surface of the absorbent article, along a length of the absorbent article, the moisture monitoring system comprising:
a sheet comprising a first surface and a second surface;
a sensor system comprising printed sensors comprising conductive traces, the sensor system being provided onto a first part of the first surface of the sheet; and
a measurement unit comprising electrical interconnections for connecting the sensor system with the measurement unit.

15. The moisture monitoring system of claim 14, further comprising an adhesive applied onto a second part of the first surface of the sheet, for attaching the moisture monitoring system onto the absorbent article.

16. The moisture monitoring system of claim 14, wherein the sensor system comprises moisture sensors.

17. The moisture monitoring system of claim 14, wherein the measurement unit fits within the first surface of the sheet.

18. The moisture monitoring system of claim 14, wherein the measurement unit fits within a central area of the first surface of the sheet.

19. The moisture monitoring system of claim 14, wherein the measurement unit covers at least part of a central area of the first surface of the sheet and covers the sensor system with which the measurement unit is being connected by means of the electrical interconnections.

20. The moisture monitoring system of claim 14, wherein the sheet comprises polyethylene, polypropylene, or polyethylene terephthalate.

21. The moisture monitoring system of claim 15, wherein the first part of the first surface of the sheet is within a central area of the first surface, and/or the second part of the first surface of the sheet is at an outer edge of the first surface.

22. The moisture monitoring system of claim 15, wherein the adhesive comprises hot melt glue.

23. An absorbent article comprising:
an outer surface; and
a moisture monitoring system according to claim 15 attached to the outer surface.

24. The absorbent article of claim 23, wherein the adhesive is applied at least in part onto the second part of the first surface of the sheet, the second part being at an outer edge of the first surface of the sheet, whereby the absorbent article comprises a pocket encapsulating the measurement unit.

25. The absorbent article of claim 23, wherein the absorbent article is a diaper, an incontinence pad, a sanitary napkin, a towel, or a hygienic cloth.

26. A method for applying the moisture monitoring system according to claim 15 onto an outer surface of an absorbent article, along a length of the absorbent article, the method comprising:
(i) providing a sheet comprising a first surface and a second surface;
(ii) providing a sensor system onto a first part of the first surface of the sheet, the sensor system comprising printed conductive traces;
(iii) applying an adhesive onto a second part of the first surface of the sheet;
(iv) connecting a measurement unit to the sensor system by electrical interconnections of the measurement unit; and
(v) connecting the adhesive with the outer surface of the absorbent article.

27. The method of claim 26, further comprising:
(vi) covering with a cover layer the moisture monitoring system and a periphery of the moisture monitoring system on the absorbent article.
